(19) United States
(12) Patent Application Publication
(10) Pub. No.: US 2025/0213329 A1
(43) Pub. Date: Jul. 3, 2025

(54) APPARATUSES AND METHODS FOR ORTHODONTIC TREATMENT WITH PREFABRICATED ATTACHMENTS

(71) Applicant: Align Technology, Inc., San Jose, CA (US)

(72) Inventors: Avi KOPELMAN, Palo Alto, CA (US); Jun SATO, San Jose, CA (US)

(21) Appl. No.: 19/008,533

(22) Filed: Jan. 2, 2025

Related U.S. Application Data
(60) Provisional application No. 63/617,074, filed on Jan. 2, 2024.

(51) Int. Cl. A61C 7/00; A61C 7/08

(52) U.S. Cl. CPC A61C 7/002; A61C 7/08

(57) ABSTRACT

Methods and apparatuses for orthodontic treatment with prefabricated attachments. The prefabricated attachments may have predetermined characteristics, such as shape, size, rigidity, elasticity, deformability, material, and/or color. A treatment plan and aligners for moving the patient's teeth may be based on using the prefabricated attachments. In some examples, the prefabricated attachments are bonded on the patient's teeth, and a scan of the patient's teeth is made with the prefabricated attachments bonded to the patient's teeth. In some examples, a recommended set of prefabricated attachments based on the patient's malocclusions or other factors is provided.

### CLAIM OF PRIORITY

This patent application claims priority to U.S. provisional patent application No. 63/617,074, titled “APPARATUSES AND METHODS FOR ORTHODONTIC TREATMENT WITH PREFABRICATED ATTACHMENTS,” and filed on Jan. 2, 2024, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present disclosure generally relates to orthodontic treatments using prefabricated dental attachments. More particularly, the present disclosure is related to systems, devices, and methods for treatment planning and design and fabrication of dental aligners based on prefabricated dental attachments.

### BACKGROUND

Orthodontics is a dental specialty in which a patient's teeth are moved to positions where function and/or aesthetics are optimized. Traditionally, appliances such as braces are applied to a patient's teeth by a dental practitioner and the set of braces exerts continual force on the teeth and gradually urges them toward their intended positions. Over time and with a series of clinical visits and adjustments to the braces, the dental practitioner adjusts the appliances to move the teeth toward their final destination.

More recently, alternatives to conventional orthodontic treatment with traditional affixed appliances (e.g., braces) have become available. For example, systems including a series of preformed aligners have become commercially available from Align Technology, Inc., San Jose, Calif., under the tradename Invisalign® System. The Invisalign® System is described in numerous patents and patent applications assigned to Align Technology, Inc. including, for example in U.S. Pat. No. 6,450,807, as well as on the company's website, which is accessible on the World Wide Web (see, e.g., the URL “align.com”). The Invisalign® System includes designing and/or fabricating multiple, and sometimes all, of the aligners to be worn by the patient before the aligners are administered to the patient and used to reposition the teeth (e.g., at the outset of treatment).

It would be particularly beneficial to provide improved techniques and orthodontic systems that make orthodontic treatment planning simpler and more flexible for dental practitioners. It would also be beneficial to provide improved techniques and orthodontic systems that improve the efficiency of the treatment planning and appliance fabrication process.

### SUMMARY OF THE DISCLOSURE

Described herein are methods and apparatuses (e.g., systems, devices, software) for treatment planning and design and fabrication of dental appliances (e.g., aligners) based on prefabricated dental attachments having predetermined characteristics. The predetermined characteristics may include shape, size, rigidity, elasticity, deformability, material, color (e.g., shade) and/or other characteristics of a dental attachment. A treatment plan may determine the best staging using selected attachments based on a patient's current tooth configuration and target tooth configuration.

Shell aligners and aligner systems may use one or more dental auxiliaries (including tooth attachments) bonded to the surface of a tooth to help secure the shell aligner to the teeth and/or to assist in applying forces to achieve a desired tooth movement. The force applied to the teeth may at least partially arise from the aligner to which the attachments are engaged. This force may result from the elastic nature of the materials forming the aligner.

Orthodontic treatment planning may include the collection and processing of images of the patient's teeth. These images may be used to determine the current configuration of the patient's teeth and as a basis for determining a final tooth configuration representing a desired final outcome. Treatment planning software may be used to generate a treatment plan in which the teeth are incrementally moved over multiple stages, with each stage typically associated with a corresponding aligner. Treatment planning may involve determining custom shapes and sizes of the dental auxiliaries (e.g., attachments) and aligners for applying forces on the teeth according to the treatment plan.

In any of the methods and apparatuses described herein the template (e.g., jig) for receiving, positioning and assisting in securing the prefabricated aligners to the patient's teeth may be customized, even while using prefabricated (un-customized) attachments. For example, any of these methods and apparatuses may include digitally forming the patient-specific (e.g., customized) jig, which may be based on the digital model (e.g. intraoral scan) of the patient's teeth, and fabricated a custom template from the digital model using any appropriate fabrication technique, including 3D printing or the like. Any of these methods may include inserting or otherwise coupling the prefabricated attachments to the template, and/or applying the template to the patient's teeth and/or positioning/securing the prefabricated attachments to the patient's teeth using the jig.

According to one example, a method of forming a series of aligners for orthodontic treatment of a patient's dentition includes: accessing a library of prefabricated dental attachments, wherein the prefabricated dental attachments have predetermined characteristics; generating an orthodontic treatment plan for moving the patient's teeth from an initial configuration toward a target configuration by applying repositioning forces to the patient's teeth according to a series of treatment stages; selecting one or more prefabricated dental attachments from the library of prefabricated dental attachments for applying the repositioning forces to the patient's teeth according to the series of treatment stages; determining a shape of each aligner of the series of aligners, wherein each aligner is configured to apply repositioning forces to the patient's teeth according to a corresponding stage of the orthodontic treatment plan, further wherein one or more of the aligners includes one or more attachment cavities to accommodate the selected one or more prefabricated dental attachments; and causing the series of aligners to be fabricated based on the determined shapes. The prefabricated dental attachments may have different characteristics including one or more of: a shape, a size, a rigidity, an elasticity, a deformability, and a material. The method may further include providing a recommendation as to which of the prefabricated dental attachments to select from the library based at least partially on the initial configuration and the target configuration of the patient's teeth. The method may further includes displaying a user interface that allows a user to enter one or more types of malocclusions, one or more chief concerns, or one or more types of malocclusions and one or more chief concerns, and further comprising providing a recommendation as to which of the prefabricated dental attachments to select based at least partially on information entered in the user interface. The one or more prefabricated attachments are configured to be bonded at one or more predetermined locations on the patient's teeth. The method may further include receiving a scan of the patient's teeth with the selected one or more prefabricated dental attachments bonded to the patient's teeth, wherein the shape of each aligner is based on the scan. The method may further include receiving a scan of the patient's teeth without the selected one or more prefabricated dental attachments bonded to the patient's teeth, wherein the orthodontic treatment plan is generated based on the scan. The method may further include determining attachment bonding zones corresponding to approximate bonding locations of the selected one or more prefabricated dental attachments on the patient's teeth, and providing the attachment bonding zones to a user. The method may further include fabricating the series of aligners based on the instructions. The method may further include generating a virtual attachment placement template device (e.g., “jig”) having a shape configured to fit on the patient's teeth and position the selected one or more dental attachments at corresponding bonding locations of the patient's teeth. The method may further include transmitting instructions for fabricating an attachment placement template device based on the virtual attachment placement template device, wherein the attachment placement template device includes the selected one or more dental attachment attached thereto.

According to another example, a system for forming a series of aligners for orthodontic treatment of a patient's dentition includes: one or more processors; and one or more memory stores coupled to one or more processors, the one or more memory stores configured to store computer instructions that, when executed by the one or more processors, perform a computer-implemented method including: accessing a library of prefabricated dental attachments, wherein the prefabricated dental attachments have predetermined characteristics; generating an orthodontic treatment plan for moving the patient's teeth from an initial configuration toward a target configuration by applying repositioning forces to the patient's teeth according to a series of treatment stages; selecting one or more prefabricated dental attachments from the library of prefabricated dental attachments for applying the repositioning forces to the patient's teeth according to the series of treatment stages; determining a shape of each aligner of the series of aligners, wherein each aligner is configured to apply repositioning forces to the patient's teeth according to a corresponding stage of the orthodontic treatment plan, further wherein one or more of the aligners includes one or more attachment cavities to accommodate the selected one or more prefabricated dental attachments; and causing the series of aligners to be fabricated based on the determined shapes. The computer-implemented method may further include: displaying a user interface that allows a user to enter one or more types of malocclusions, one or more chief concerns, or one or more types of malocclusions and one or more chief concerns; and providing a recommendation as to which of a library of multiple prefabricated dental attachments to select based at least partially on information entered in the user interface. The computer-implemented method may further include providing a recommendation as to which of the prefabricated dental attachments from the library to select based at least partially on the initial configuration and the target configuration of the patient's teeth. The predetermined characteristics of the selected one or more dental attachments may include one or more of: a shape, a size, a rigidity, an elasticity, a deformability, a material, and a color of the selected one or more dental attachments. The computer-implemented method may further include receiving a scan of the patient's teeth with the selected prefabricated one or more dental attachments bonded to the patient's teeth at the bonding locations, wherein the shape of each aligner is based on the scan. The computer-implemented method may further include receiving a scan of the patient's teeth without the selected prefabricated one or more dental attachments bonded to the patient's teeth, wherein the orthodontic treatment plan is generated based on the scan. The computer-implemented method may further include generating a virtual attachment placement template device having a shape configured to fit on the patient's teeth and position the selected prefabricated one or more dental attachments at corresponding bonding locations of the patient's teeth. The computer-implemented method may further include transmitting instructions for fabricating an attachment placement template device based on the virtual attachment placement template device, wherein the attachment placement template device includes the selected prefabricated one or more dental attachment attached thereto.

According to a further example, a method of aligning a patient's teeth, the method includes: receiving a treatment plan for moving the patient's teeth, the treatment plan comprising a plurality of treatment stages based on applying forces on the patient's teeth, wherein the forces are applied by a series of aligners at locations on the patient's teeth corresponding to dental attachments bonded on the patient's teeth; bonding prefabricated dental attachments at the locations on the patient's teeth according to the treatment plan; scanning the patient's teeth with the prefabricated dental attachments bonding on the patient's teeth; generating at least one of the series of aligners based on the scan, wherein the at least one of the series of aligners includes one or more attachment cavities configured to engage with one or more of the prefabricated dental attachments for applying forces on the patient's teeth according to the treatment plan. The method may further include selecting the prefabricated dental attachments from a library dental attachments after the treatment plan is generated. The method may further include generating an attachment placement template device having a shape configured to fit on the patient's teeth and position the prefabricated dental attachments at corresponding bonding locations of the patient's teeth. The prefabricated dental attachments have different characteristics including one or more of: a shape, a size, a rigidity, an elasticity, a deformability, a material, and a color. The method may further include providing a recommendation as to which of multiple prefabricated dental attachments in a library to select based at least partially on an initial configuration and a target configuration of the patient's teeth.

According to an additional example, a system for forming a series of aligners for orthodontic treatment of a patient's dentition includes: one or more processors; and one or more memory stores coupled to one or more processors, the one or more memory stores configured to store computer instructions that, when executed by the one or more processors, perform a computer-implemented method including: receiving a treatment plan for moving the patient's teeth, the treatment plan comprising a plurality of treatment stages based on applying forces on the patient's teeth, wherein the forces are applied by a series of aligners at locations on the patient's teeth corresponding to dental attachments bonded on the patient's teeth; bonding prefabricated dental attachments at the locations on the patient's teeth according to the treatment plan; scanning the patient's teeth with the prefabricated dental attachments bonding on the patient's teeth; generating at least one of the series of aligners based on the scan, wherein the at least one of the series of aligners includes one or more attachment cavities configured to engage with one or more of the prefabricated dental attachments for applying forces on the patient's teeth according to the treatment plan. The computer-implemented method may further include selecting the prefabricated dental attachments from a library of prefabricated dental attachments after the treatment plan is generated. The computer-implemented method may further include generating an attachment placement template device having a shape configured to fit on the patient's teeth and position the prefabricated dental attachments at corresponding bonding locations of the patient's teeth. The prefabricated dental attachments may have different characteristics including one or more of: a shape, a size, a rigidity, an elasticity, a deformability, a material, and a color. The computer-implemented method may further include providing a recommendation as to which of the multiple prefabricated dental attachments to select based at least partially on the initial configuration and the target configuration of the patient's teeth.

According to another example, a method includes: receiving a treatment plan for moving a subject's teeth, wherein the treatment plan comprises a plurality of treatment stages; selecting a set of prefabricated attachments from a catalogue of prefabricated attachments; positioning the set of prefabricated attachments within an attachment placement template device, wherein the attachment placement template device positions the set of prefabricated attachments at locations on the subject's teeth based on the treatment plan; bonding the set of prefabricated attachments to the subject's teeth using the attachment placement template device; and receiving aligners based on the treatment plan for treating the subject's teeth, wherein the aligners are configured to engage with the set of prefabricated attachments bonded to the subject's teeth. Selecting the set of attachments may include selecting from a catalog of prefabricated attachments having different attachment characteristics. The method may further include fabricating the attachment placement template device. The method may further include generating a virtual attachment placement template device with a set of virtual prefabricated attachments attached thereto, and fabricating the attachment placement template device and the set of prefabricated attachments based on the virtual attachment placement template device with the attached set of virtual prefabricated attachments. The method may further include generating a virtual attachment placement template device, fabricating the attachment placement template device based on the virtual attachment placement template device, and attaching the set of prefabricated attachments to the attachment placement template device. The set of prefabricated attachments may be made of a different material than the attachment placement template device. The method may further include fabricating the attachment placement template device using direct fabrication (e.g., three-dimensional printing).

According to a further example, a system for orthodontic treatment of a patient's dentition includes: one or more processors; and one or more memory stores coupled to one or more processors, the one or more memory stores configured to store computer instructions that, when executed by the one or more processors, perform a computer-implemented method including: receiving a treatment plan for moving a subject's teeth, wherein the treatment plan comprises a plurality of treatment stages; selecting a set of prefabricated attachments from a catalogue of prefabricated attachments; generating a virtual attachment placement template device with a virtual set of prefabricated attachments attached thereto, wherein the virtual attachment placement template device is configured to position the virtual set of prefabricated attachments at predetermined locations on a three-dimensional model of the patient's teeth; and causing an attachment placement template device to be fabricated based on the virtual attachment placement template device, wherein the attachment placement template device is configured to have the selected set of prefabricated attachments attached thereto. The computer-implemented method may further include causing an aligner to be fabricated based on treatment plan, wherein the aligner is configured to engage with the selected set of prefabricated attachments bonded to the subject's teeth and to apply repositioning forces on the subject's teeth according to the treatment plan. The computer-implemented method may further include providing a recommendation for the selection of the set of prefabricated attachments from the catalogue of attachments based on one or more tooth movements of the treatment plan. The set of attachments may be made of a different material than the attachment placement template device. The attachment placement template device may be fabricated using direct fabrication (e.g., three-dimensional printing).

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A is a flowchart illustrating an example process using prefabricated dental attachments.

FIG. 1B illustrates an example diagram showing an example implementation of using prefabricated dental attachments.

FIGS. 2A-2C illustrate examples of different dental attachments.

FIG. 3 illustrates an example of an elastically deformable attachment.

FIG. 4 illustrates another example of an elastically deformable attachment.

FIGS. 5A-5B illustrate yet another example of an elastically deformable attachment.

FIG. 6 illustrates an example attachment placement template device.

FIG. 7 illustrates another example of an attachment placement template device.

FIGS. 8A and 8B illustrate yet another example of an attachment placement template device.

FIG. 9 is a flowchart illustrating an example process using prefabricated dental attachments, including fabricating an attachment placement template device.

FIG. 10A illustrates examples of Facial Axis of the Clinical Crown (FACC) lines of teeth of a dentition.

FIG. 10B illustrates examples of facial axis points (FA) of teeth of a dentition.

FIG. 11 is a flowchart illustrating an example process using prefabricated dental attachments and predetermined attachment bonding locations.

FIG. 12 is a flowchart illustrating an example process using prefabricated dental attachments, where a treatment plan is created and optionally updated.

FIG. 13 is a flowchart illustrating an example process using prefabricated dental attachments, where the types of attachments are predicted, and the attachments are bonded at recommended zones of the teeth.

FIG. 14 illustrates an example block diagram of an example system that includes one or more devices for implementing one or more of the methods described herein.

FIG. 15 is a diagram of one example of a computing environment within which the methods and apparatuses described herein may be implemented.

FIG. 16 is a schematic illustration of one example of an attachment modeling engine as described herein.

### DETAILED DESCRIPTION

Described herein are apparatuses (e.g., systems and devices, including software) and methods of orthodontic treatment planning that include using prefabricated (also referred to as “off-the-shelf”) attachments. A prefabricated attachment refers to an attachment that is already made. This is in contrast to conventional attachments in which the attachments are customized based on the treatment plan, then made after the treatment plan has been established. The characteristics of refabricated attachments are already determined and therefore need not be customized based on a particular treatment plan. The characteristics of the prefabricated attachments may include shape, size, rigidity, elasticity, deformability, material, color (e.g., shade) and/or other characteristics of a dental attachment.

Prefabricated attachments can provide a number of advantages. For example, whereas conventionally both the attachments and the aligners are customized, only the aligners may need to be customized. For instance, the shape of the aligners may be customized based on the characteristics of the prefabricated attachments and their intended positions on the patient's teeth according to the treatment plan. In some cases, an attachment placement template device is fabricated and configured to place the off-the-shelf attachments at proper locations on the teeth according to the treatment plan. The attachment placement template device may be designed using the known characteristics of the prefabricated attachments and a known shape of the tooth/teeth (e.g., from an intraoral scan).

In some cases, the attachments are already fabricated (prefabricated) and available for the dental practitioner to use. Thus, the dental practitioner may bond the prefabricated attachments on the patient's teeth and scan the patient's teeth with the bonded attachments. A digital 3D model may be generated based on the scan and used to determine the shape of the aligners in accordance with the treatment plan. This allows more flexibility as to the positions of the attachments on the patient's teeth. For example, the attachments positions may not need to be as precise compared to when the treatment plan dictates the attachment positions prior to placement. Therefore, there may be no need to correct the position of the attachments on the patient's teeth. In addition, there may be no need for an attachment placement template device. Instead, the dental practitioner may be instructed to bond the attachments at particular locations, such as at or near a center of a crown surface. Additionally, since the scan of the patient's teeth is taken with the attachments already attached, and the treatment plan and aligners are calculated based on the scan, there may be no need to take an additional scan of the patient's teeth. Further, the attachment locations may be based on standard locations or based on landmarks on a tooth, which may make it easier for the dental practitioner to re-bond the attachments to the teeth if debonding occurs during the course of treatment. In addition, the treatment plan may be generated only once (e.g., no need to adjust treatment plan based on actual placement of the attachments).

The prefabricated attachments may be categorized based on one or more characteristics. For example, some attachments may have particular shapes that are conducive to providing certain tooth movements, such as translation, rotation, intrusion, and/or extrusion. Some attachment shapes may perform better on certain tooth types, such as molar, premolar, incisor, and/or canine teeth. Some attachments may be rigid and inflexible, while other attachments may have some flexibility and/or elasticity. Some attachment colors may be better suited for certain teeth (e.g., anterior or posterior teeth) and/or may be color-matched (e.g., shaded) with the patient's teeth. For example, attachments may be available in all tooth shades so that a best shade for a particular patient may be available. In some cases, attachments having non-tooth colors (e.g., vivid or patterned colors) may be available.

In some examples, a device (e.g., computer) may have a user interface to present and/or receive information from a user (e.g., dental practitioner). For example, the device may present a catalogue (also referred to as a library) of attachments. The attachments may be presented by category. In some cases, the device is configured to provide a recommendation as to which attachments to use for a particular patient's treatment. The recommendation may be determined based on the type(s) of malocclusion, a target tooth configuration, chief concerns, and/or other factors.

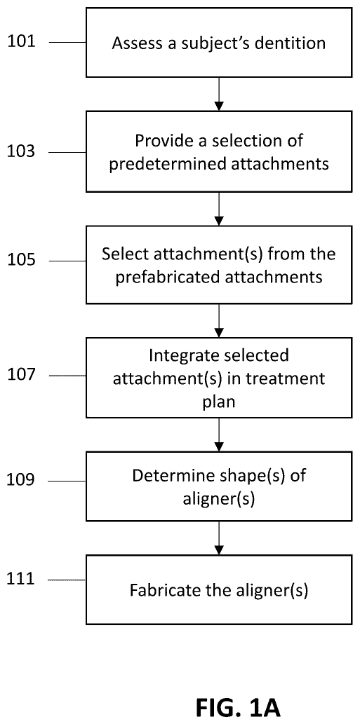

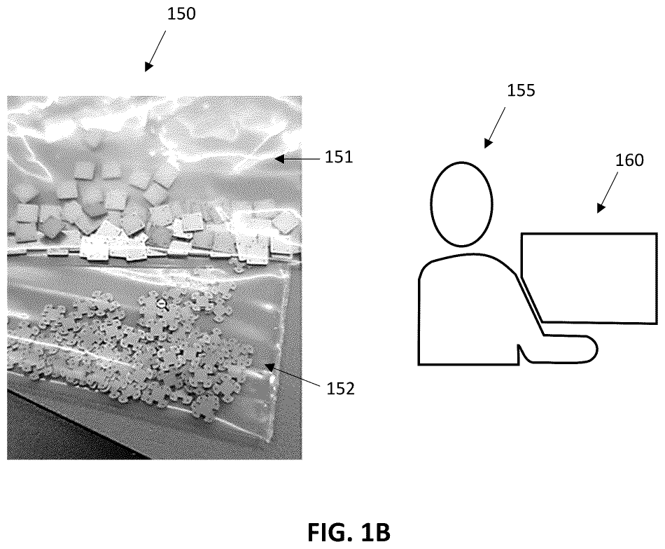

FIG. 1A is a flowchart illustrating an example process using prefabricated dental attachments. At 101, a subject's dentition is assessed. This may include obtaining the type(s) of malocclusion (e.g., class I, class II, class III, crowding, overbite, underbite, overjet, crossbite, open bite, gapped teeth, etc.), chief concerns of the subject, and/or other factors related to the current condition and a target condition of the subject's teeth. The subject's dentition may include obtaining one or more images of the subject's mouth, representing a current configuration of the subject's teeth. In some examples, this involves using an intraoral scanner configured to obtain multiple images of the subject's intraoral cavity. In some cases, one or more images are obtained by taking pictures of the subject's teeth using a camera that is not an intraoral scanner, such as a camera of a person device (e.g., smart phone). The image(s) that are collected (by scanner or other camera) may be used to generate a three-dimensional (3D) digital model of the subject's teeth. The 3D digital model may be used along with other information (e.g., type of malocclusion(s), chief concerns, subject's choice of treatment type and duration, etc.) for determining a target configuration of the patient's teeth.

The assessment may include generating a treatment plan for treating one or more malocclusions and/or chief concerns of the subject. The treatment plan may be based on the 3D digital model of the subject's teeth. For example, the 3D digital model based on the one or more images/scans of the subject's teeth in a current configuration may be used as an initial configuration. The plan may include generating a series of stages in which the subject's teeth are incrementally moved from the initial configuration toward a target configuration by applying repositioning forces to the subject's teeth. Each of the stages can be implemented by applying forces on one or more of the subject's teeth. The forces may be directed in predetermined direction(s) and/or with a predetermined amount of force to move the one or more teeth toward a sub-target configuration for each stage. Each of the stages of the treatment plan can be associated with a corresponding dental appliance (e.g., aligner). The aligners may be shell aligners (e.g., made of polymer(s)) that are configured to resiliently apply the forces to the subject's teeth according to a corresponding stage of the treatment plan.

The assessment may include determining whether it would be beneficial to use one or more attachments to achieve particular types of orthodontic tooth movements. Orthodontic tooth movements may include, for example, tipping, uprighting, translation, intrusion, extrusion, rotation, torquing, etc. Some tooth movements may be adequately accomplished with an aligner without the use of an attachment on the subject's teeth. Other tooth movements may require the use of an attachment to accomplish a desirable outcome. Other tooth movements may optionally use an attachment, for example, to accomplish a desired tooth movement at a faster rate than without the use of an attachment. Whether an attachment could/should be used may also be determined based on other factors such as the type of tooth (e.g., incisor, premolar, canine, molar), the location of a tooth (e.g., upper, lower, tooth number), the direction of movement (e.g., mesiodistal, buccolingual, extrusion, intrusion, etc.), and/or the presence of adjacent teeth.

At 103, a selection of prefabricated attachments is provided. The prefabricated dental attachments have already been made or designed and ready to make. Therefore, the prefabricated attachments have known characteristics. The characteristics may include, for example, a shape, a size, a rigidity, an elasticity, a deformability, a material, a color and/or other characteristics of the attachment. Different types of prefabricated attachments have different characteristics based, for example, on the type of orthodontic tooth movement and/or which tooth they are bonded to. For example, an attachment used for applying an extrusion force may have a different size and shape than an attachment used for applying a translation force. Attachments having a particular rigidity, elasticity, deformability and/or material may be conducive to providing certain types of movement and/or applying a certain amount of force. It may be desirable for an attachment for an anterior tooth to have a certain color and/or be made of a certain material since anterior teeth are generally the most visible.

The prefabricated dental attachments may be available as a catalog of attachments. The catalog may be organized based on the characteristics of the attachments and/or one or more other factors. For example, the catalog may organize the attachments based on their shape, size, rigidity, elasticity, deformability, material, color and/or other characteristics. Alternatively or additionally, the catalog may organize the attachments based on the types of tooth movement(s) and/or the types of tooth correction that the attachments are typically used for. In some examples, the catalogue is presented in a user interface, as described herein.

In some cases, a recommendation for one or more of the dental attachments is optionally provided. The recommendation may be based on the type of tooth movement and/or type of correction, as discussed above. The recommendation may be determined based on a magnitude and/or direction of force to be applied to one or more teeth, a complexity of movement of the one or more teeth, an expected location of the attachment (e.g., anterior or posterior tooth), a desired thickness of an aligner that engages with the attachment, a duration in which force is to be applied to the attachment by the aligner(s), and/or other factors. In some cases, the recommendation may be provided to the user via the user interface.

At 105, one or more attachments from the selection of prefabricated dental attachments is selected. In some cases, the selection is received from the user (e.g., from a user interface). In other cases, the selection is made automatically (e.g., based on recommendation(s) determined at 103). A set of attachments for implementing one or more stages of the treatment plan may be selected.

At 107, the selected attachment(s) are integrated into the treatment plan. This may involve determining which of the selected attachment(s) should be placed on which tooth, and where on the tooth/teeth the selected attachment(s) should be placed, at each appropriate stage of the treatment plan. In some examples, this may involve using virtual modeling. For example, a virtual 3D model the selected attachment(s) may be placed on a virtual 3D model of the subject's teeth at particular stages of the treatment plan.

At 109, the shape(s) of one or more aligners for implementing the treatment plan are determined. For example, one or more virtual aligners may be generated based on applying forces on the initial 3D digital model or an intermediate 3D digital model according to a corresponding stage of the treatment plan. The aligner(s) may include tooth cavities or recesses for accepting the subject's teeth. In addition, the shape of the aligner(s) may include one or more attachment cavities that are configured to accept corresponding one of more selected attachments at particular locations relative to the teeth for applying the forces on the teeth. The attachment cavities may be within the tooth cavities, as discussed herein.

In some cases, simulated forces (e.g., mimicking forces applied by an aligner) are applied to the virtual 3D attachment(s) on the virtual teeth. In some examples, this may be used to verify that the selected attachment(s) apply appropriate forces for achieving the desired tooth movement(s) (e.g., toward the sub-target configuration for each stage). If the it is determined that the selected attachment(s) do not result in a desired tooth movement, aspects of the virtual aligner, such as a shape, thickness and/or rigidity of one or more regions of the virtual aligner, may be modified until the desired tooth movement is achieved. Additionally or alternatively, a position of the selected attachment(s) on the tooth/teeth may be modified until the desired tooth movement is achieved. In some examples, the treatment plan is modified to achieve the desired tooth movement(s).

At 111, one or more aligners are fabricated based on the shape(s) of the one or more virtual aligners. In some cases, a series of aligners are fabricated for implementing the series of stages of the treatment plan. Each aligner of the series of aligners is configured to apply repositioning forces to the patient's teeth according to a corresponding stage of the treatment plan. The shape of each aligner can be based on characteristics of a corresponding selected one or more attachments and bonding locations of the corresponding selected one or more attachments on the patient's teeth.

FIG. 1B shows an example implementation using prefabricated dental attachments. A user 155 is able to select one or more prefabricated attachments 150 that are already made for purchase from available stock and/or ready to make based on an existing design. For example, the prefabricated attachments may include a first type of attachment 151 and a second type of attachment 152. In this example, the first and second attachments 151 and 152 have different shapes. The user 155 may select the one or more attachments from a catalogue of attachments presented to the user via a user interface 160. The user interface 160 may present the attachments 150 organized based on characteristics of the attachments. For example, the attachments may be displayed based on their shape, size, rigidity, elasticity, deformability, material, color and/or other characteristics. Alternatively or additionally, the attachments may be displayed based on the types of tooth movement(s) and/or the types of tooth correction that the attachments may be used for. In some cases, the user interface may be interactive in that a user may be able to search and/or sort the attachments based on or more factors. The user interface 160 may list the various types of attachments and their availability and/or expected delivery date. In some cases, the user interface 160 may display renditions (e.g., pictures and/or drawings) of the attachments.

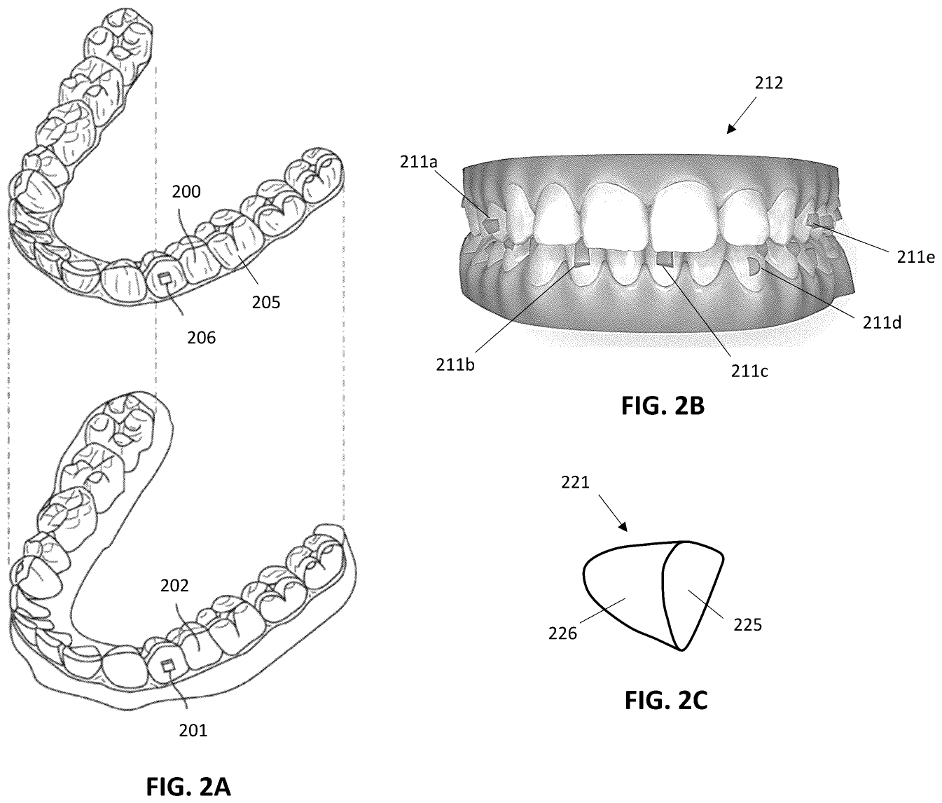

The dental attachments may have any of a number of predetermined shapes and sizes. FIGS. 2A-2C show examples of different dental attachments. FIG. 2A shows an example attachment 201 bonded to a tooth of a patient's dentition 202, and an aligner 200 that is configured to fit on the dentition 202. The aligner 200 has multiple tooth cavities 205 that are shaped to accept corresponding teeth of the dentition 202. The aligner 200 also has an attachment cavity 206 that is shaped to accept the attachment 201 when the aligner 200 is placed on the dentition 202. For example, one or more walls of the aligner 200 that define the attachment cavity 206 may be configured to apply a force on the attachment 201 in a prescribed direction and magnitude according to a corresponding stage of a treatment plan. In this case, the attachment cavity 206 is within one of the tooth cavities 205. The aligner 200 may be made of a polymer material (e.g., clear polymer).

FIG. 2B shows an example of a dentition 212 having multiple attachments bonded to different teeth. The attachments have different shapes and sizes. For example, attachments 211a, 211b, 211c, and 211e have rectangular shapes of different sizes, and attachment 211d has a curved shape. Some methods involve forming the attachments to have customized shapes and sizes. For example, the attachments may have customized shapes and sizes based on optimizing the forces placed on the teeth.

The dental attachments can include engagement surfaces that are configured to engage with corresponding one or more aligners. For example, FIG. 2C shows an example attachment 221. The attachment 221 is configured to protrude from a tooth when bonded to the tooth. An engagement surface 225, which in this case is a flat surface, is configured to be at a predetermined angle with respect to the surface of the tooth such that, when one or more aligners engage with the attachment 221, a prescribed force is applied to the tooth according to one or more stages of the treatment plan. In this example, the attachment 221 has a curved surface 226 that may be inactive with respect to providing forces against the tooth.

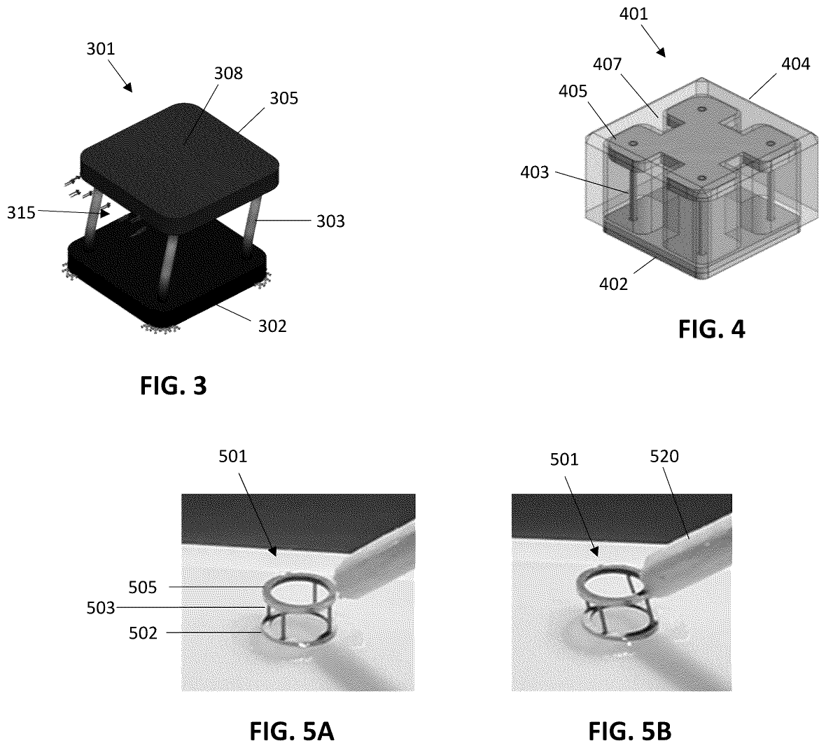

FIGS. 3-5B show example dental attachments that have elasticity and/or are deformable. FIG. 3 shows an example attachment 301 that includes an apex region 305 and a parallel base region 302 separated by an interface region that includes multiple gaps or openings 315 spanned by the four elastically deformable biases 303. The base region 302 may have an interface surface that is configured to be bonded to a tooth surface. In some cases, the interface surface has a customized shape to correspond to a shape of a particular type of tooth surface (e.g., molar, canine, incisor). In some examples, the base region 302 has a customized shape while the apex region 305 and the biases 303 are not customized based on tooth type.

During operation, force may be applied from an aligner to displace the apex region 305 relative to the base region 302 by contact with the aligner on an engagement surface 308 of the apex region 305. FIG. 3 illustrates how the attachment 301 can be deflected by pushing against the engagement region 308 of the apex 305 (e.g., top and/or sides). In some examples, the biases 303 are made of a shape memory material, such as Nitinol.

FIG. 4 shows another example of an attachment 401 that is also configured to deform. Attachment 401 includes an apex region 405 and base region 402 connected by elastically deformable biases 403, where the apex region 405 is configured to deflect relative to the base region 402, similar to the attachment 301 of FIG. 3. In this example, the shapes of the apex region 405 and the base region 402 have cutouts 407 (e.g., instead of the square shapes of the apex region 305 and the base region 302 of attachment 301). The attachment 401 also includes a cover 404 that covers the gaps between the apex region 405 and base region 402. This may prevent fluid (e.g., saliva) from entering the gaps. Force may be applied to the top and/or sides of the cover 404 to cause the deformation as described above with respect to attachment 301. In this example, the cover 404 has a rectangular (e.g., rectangular prism) shape. However, the cover 404 can have any of a number of shapes (e.g., triangular, round, ellipsoid, etc.). In some examples, the cover 404 may be made of polymer material (e.g., rigid polymer).

FIGS. 5A and 5B shows another example an attachment 501 that is also configured to deform. The attachment 501 includes an apex region 505 and base region 502 connected by elastically deformable biases 503, where the apex region 505 is configured to deflect relative to the base region 402, similar to the attachments 301 and 401. FIG. 5A shows the attachment 501 prior to being deformed, and FIG. 5B shows the attachment 501 when a force is applied by an object 520 on an engagement surface of the apex region 505, thereby causing the apex region 505 to deflect relative to the base region 502. In the example of attachment 501, the apex region 501 has a round shape with a central opening. A cover may be added to the attachment 501 to cover the various gaps and to prevent fluid from entering the gaps. Such a cover may have a round shape to match the round shaped apex region 505 and base region 402. Alternatively, a different shaped cover may be used (e.g., rectangular, ellipsoid, triangular, etc.).

In some examples, the attachment may be a “universal” attachment that is configured to provide any type of tooth movement. A treatment plan and aligners may be designed around using such universal attachments. A universal attachment may be configured to apply a prescribed force in a prescribed direction and magnitude based on its location on a tooth and the expected force applied by the aligner. In some cases, a universal attachment may have some elasticity such that it can deform when force from an aligner is applied to it. In some cases, the attachment may deform differently based on the direction of the force from the aligner.

The attachments described herein may an any type of device that is configured to be attached (e.g., bonded) to one or more teeth. The attachments may be configured to actively engage with one or more aligners to apply forces to the teeth. In other cases, the attachments may be configured to passively interact with one or more aligners. For example, in some cases, the attachment may be a bite ramp that is configured to adjust a patient's bite. In some examples, a bite ramp may be attached to a lingual side of an incisor to correct a deep bite. The aligner(s) may have corresponding cavities or openings to accommodate the bite ramps. In other cases, the aligners themselves have bite ramps.

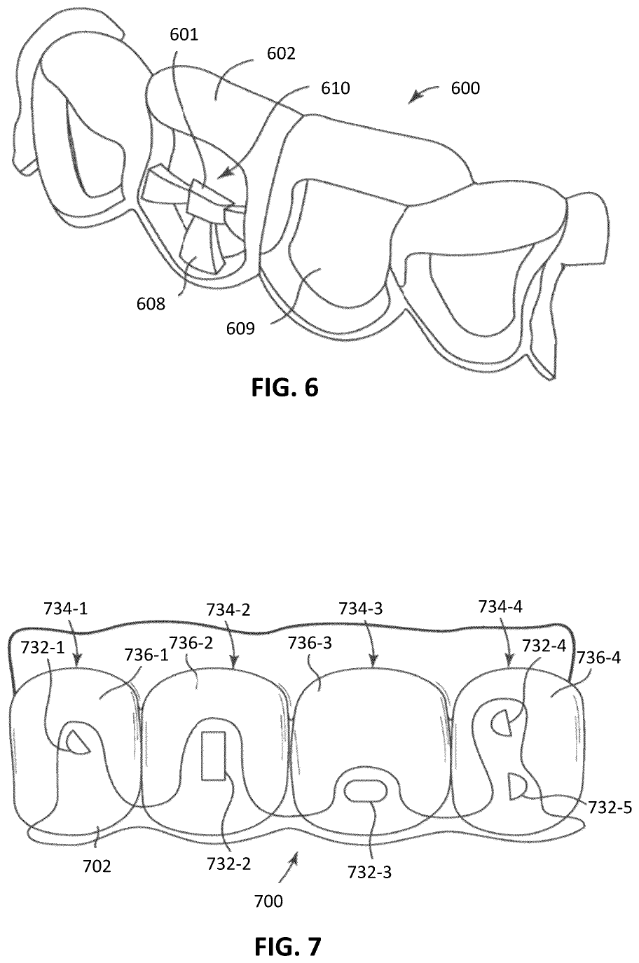

In some cases, the attachments are positioned with respect to the teeth using an attachment placement template device. The attachment placement template device may be fabricated separately from the attachments. In some cases, the attachment placement template device is made of one or more different materials than the attachment placement template device. Examples of attachment placement template devices are described in U.S. Patent Application Publication 2022/0183795, published on Jun. 16, 2022, which is incorporated by reference in its entirety herein. FIGS. 6-8B show examples of different types of attachment placement template devices.

FIG. 6 shows an example attachment placement template device 600. The attachment placement template device 600 includes a body 602 that is shaped to contact at least some surfaces of the patient's teeth to align the attachment 601 in a predetermined position with respect to a corresponding tooth. The predetermined position for the attachment 601 may be based on the treatment plan, as described herein. The attachment 601 is positioned within an aperture 610 of the body 602. Supports 608 are coupled with the attachment 601 and the body 602 to support the attachment 601 at the particular position with respect to a tooth when the body 602 is placed on the patient's teeth. A bonding surface of the attachment 601 may include a bonding material (e.g., adhesive) to bond the attachment 601 to the corresponding tooth. In some examples, the bonding material is activated (e.g., by light (e.g., UV) and/or pressure). In this example, there are three support 608; however, any number of supports may be used to support the attachment 601 in place (e.g., 1, 2, 3, 4, 5, 6, or more). In this example, the attachment placement template device 600 includes additional openings 609 such that the body 601 does not cover some surfaces of adjacent teeth.

FIG. 7 shows another example of an attachment placement template device 700. In this example, a body 702 of the attachment placement template device 700 includes attachment cavities 732-1, 732-2, 732-3 and 732-4 that are shaped and sized to accommodate corresponding attachments. The attachment may be placed within the attachment cavity 732-1, 732-2, 732-3 and 732-4 prior to placing the body 702 on the patient's teeth. The body 702, with the attachment, may be placed on the patient's teeth to position the attachments with respect to corresponding teeth 734-1, 734-2, 734-3 and 734-4, and the attachments may be bonded to corresponding surfaces 736-1, 736-2, 736-3 and 736-4 of the teeth 734-1, 734-2, 734-3 and 734-4. In other examples, the attachment cavities 732-1, 732-2, 732-3 and 732-4 are openings (e.g., also referred to as windows) instead of cavities. The openings can be shaped and sized according to corresponding attachments such that attachments may fit within and bonded through the openings.

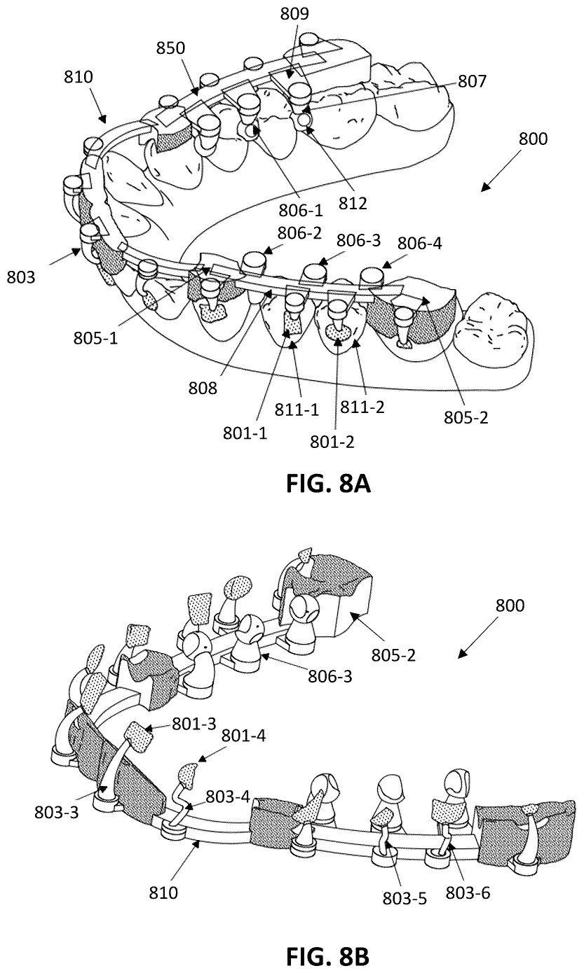

FIGS. 8A and 8B show another example of an attachment placement template device 800. In this example, the attachment placement template device includes a body 810 (e.g., beam) that is configured to extend over at least a portion of the dental arch, and that follows the shape of at least a portion of the dental arch. A number of supports (e.g., 803-3 and 803-4) that extend from the body 810 for supporting dental attachments (e.g., 801-1, 801-2, 801-3, and 801-4) at prescribed locations of corresponding teeth (e.g., 811-1 and 811-2). Retention supports (e.g., 806-1, 806-2 and 806-3) also extend off the body 810 to stabilize the attachment placement template device 800 with respect to the dental arch. The retention supports (e.g., 806-1, 806-2 and 806-3) can include contact portions (e.g., 812) that are configured to contact one or more teeth and/or gums of the dental arch. In the example shown, the contact portions (e.g., 812) are shaped and sized to contact interproximal regions between teeth. The retention supports (e.g., 806-1, 806-2 and 806-3) may include an arched or angled portion that extends the dental contact portion a distance from the frame. For example, the retention supports (e.g., 806-1, 806-2 and 806-3) may include a horizontal portion (e.g., 809) that extends in a horizontal direction with respect to the body 810, and a vertical portion (e.g., 807) that extends in a vertical direction with respect to the body 810, which position the contact portion (e.g., 812) on a surface (e.g., lingual surface) of the dental arch. Registration anchors (e.g., 805-1 and 805-2) extend from the body 810 are configured to register with some of the teeth (e.g., occlusal surfaces of the teeth) to stabilize the attachment placement template device 800 on the dental arch. In this example, the registration anchors (e.g., 805-1 and 805-2) are separated by a gap portion (e.g., 808) of the body 810 such that the gap portion (e.g., 808) spans teeth (e.g., 811-1 and 811-2). This can allow the gap portion (e.g., 808) to suspend over the dental arch and allow the body 810 to occlude less of the dental arch than an attachment placement template device that covers more tooth surfaces. For instance, the treatment professional can more easily access regions around the intervening teeth 811-1 and 811-2 for attaching the attachments 801-1 and 801-2.

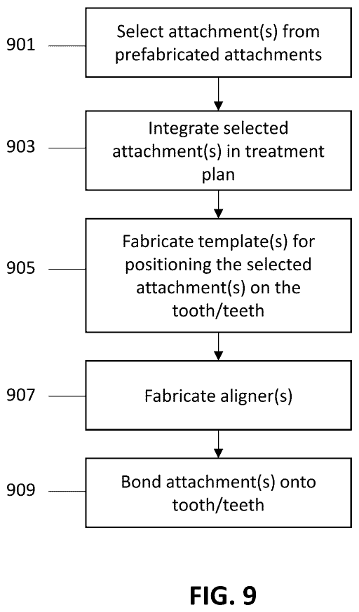

FIG. 9 is a flowchart illustrating an example process using prefabricated dental attachments, including fabricating an attachment placement template device. At 901, one or more attachments are selected from prefabricated attachments (e.g., from a catalogue of attachments). At 903, the selected attachment(s) is/are integrated into the treatment plan. As discussed herein, this can include virtual modeling and integrating the selected attachments at bonding locations on a virtual model the patient's teeth for providing repositioning forces according to the treatment plan.

At 905, one or more customized attachment placement template devices for positioning the selected attachments at the bonding locations the teeth are fabricated. In some examples, this involves creating a virtual attachment placement template device (e.g., 3D model) that fits over a 3D model of the patient's dentition at a particular stage of the treatment plan (e.g., initial or intermediate stage). The attachment placement template device may be fabricated using the virtual attachment placement template device, for example, using a molding and/or direct fabrication (e.g., 3D printing) process. The attachment placement template device may be fabricated in a dental practitioner's office or at a remote location (e.g., manufacturing facility). The attachment placement template device may be configured to couple with the selected attachments. For example, the attachment placement template device may include attachment supports (e.g., FIG. 6 or 8A-8B) and/or attachment cavities and/or openings (e.g., FIG. 7) that are configured to couple with the selected attachments.

At 907, one or more aligners are fabricated. The shape of the aligner(s) may be based on the selected attachments and the bonding location of the attachments. In some examples, the shape of the aligner(s) is determined using a virtual aligner that is configured to apply prescribed forces on the selected attachments bonded at the bonding locations on the 3D model of the patient's dentition at a particular stage of the treatment plan (e.g., initial or intermediate stage). The aligner(s) may be fabricated using the virtual aligner(s), for example, using a molding and/or direct fabrication process.

At 909, the selected attachments are bonded onto the patient's teeth/tooth at the bonding location(s). Once the attachments are attached to the teeth/tooth, the patient may use the aligner(s) to apply the prescribed forces on the teeth/tooth.

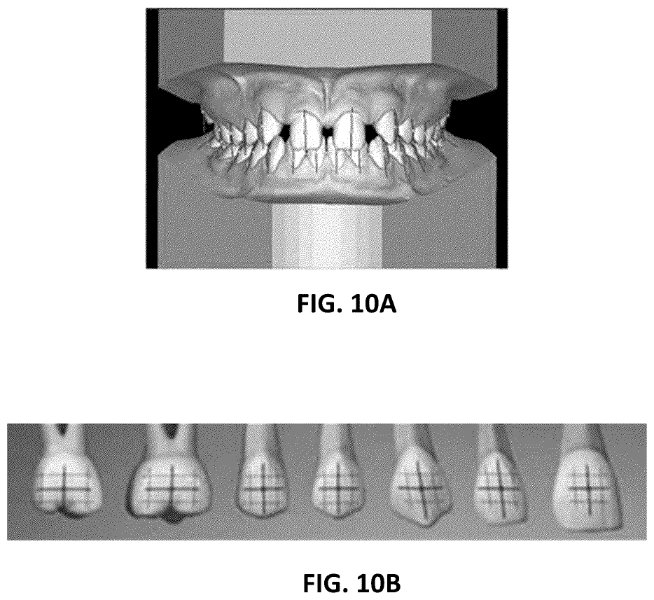

In some cases, the treatment plan is configured such that the attachments are to be bonded to the teeth at prescribed locations without the aid of a dental attachment placement structure device. That is, the attachment characteristics may be determined, and also the locations at which the attachments are bonded to the teeth may be determined, prior to generating at least part of the treatment plan. The shapes of the aligner(s) may then be designed based on the prefabricated attachments and attachment locations. In some examples, the predetermined locations may be the same for each tooth. For example, the predetermined locations may be along the Facial Axis of the Clinical Crown (FACC) of each tooth. A FACC line corresponds to the long axis of a tooth that is centered mesio-distal on the facial surface of the clinical crown, or in the case of molars, the line running down the buccal groove of the clinical crown. FIG. 10A shows examples of FACC lines. In some examples, the predetermined locations for the attachments may be at or near a facial axis point (FA) of each tooth, which refers to a point on the FACC that separates the gingival half of the clinical crown from the occlusal half of the clinical crown. FIG. 10B shows examples of FA points. In some cases, the software provides a recommended zone for bonding the attachment(s). For example, the recommended zone may be within a predetermined area around the FA points, such as shown in FIG. 10B.

In some cases, the attachments themselves may include visual markings that assist in the placement of the attachments at the predetermined locations/zones. For example, an attachment may include one or more marks (e.g., line, cross, and/or dot) that the dental practitioner may use as a reference to align the attachment with the designated tooth. For example, such mark(s) may be used as a reference to attach the attachment along a center line (e.g., FACC) or center point (e.g., FA) of the visible crown. The mark(s) may be engraved, painted, or otherwise made visible.

According to some examples, the attachments may be picked and placed onto the patient's teeth using a tool, such as a vacuum suction pen and/or tweezers. The tool may be configured to automatically pick and place the attachments or be configured to be used by a user to pick and place the attachments. The attachments may include one or more grabbable features, such as a knob, that the pen or tweezer can manipulate the attachment with. The grabbable feature may be configured to break away from the attachment once it is bonded on at tooth.

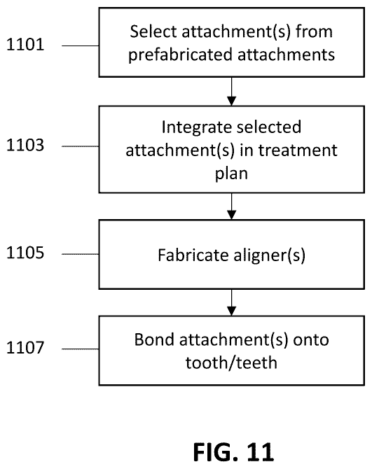

FIG. 11 is a flowchart indicating an example process using prefabricated dental attachments and predetermined attachment bonding locations. At 1101, one or more attachments are selected from prefabricated attachments (e.g., from a catalogue of attachments). At 1103, the selected attachment(s) is/are integrated into the treatment plan. The selected attachment(s) may be attached to prefabricated attachment bonding locations or zones on the teeth (e.g., at or in an area around an FA point). At 1105, one or more aligners are fabricated. The shape of the aligner(s) may be based on the selected attachments and the predetermined bonding attachment locations. At 1107, the selected attachments are bonded onto the patient's teeth/tooth at the bonding location(s). Once the attachments are attached to the teeth/tooth, the patient may use the aligner(s) to apply the prescribed forces on the teeth/tooth.

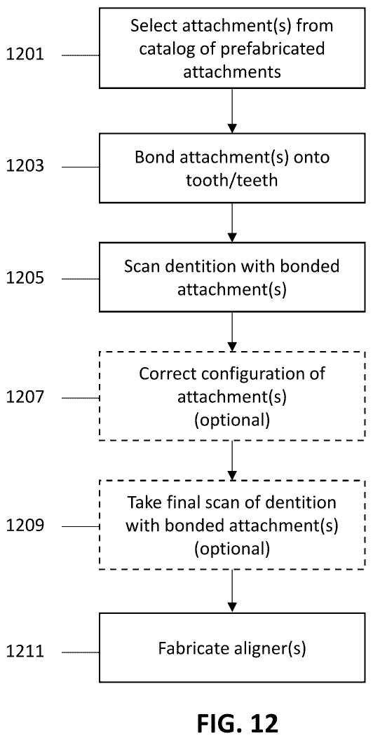

FIG. 12 is a flowchart indicating an example process using prefabricated dental attachments, where a treatment plan is created and optionally updated. At 1201, one or more attachments are selected from a catalog of prefabricated attachments. At 1203, the selected attachments are attached to the patient's teeth at locations on the patient's teeth as dictated by the treatment plan. At 1205, the patient's dentition with the attachments bonded thereto is scanned to obtain images of the dentition. The images may be used to generate a virtual 3D model of the dentition.

At 1207, the configuration of the attachments on the teeth may optionally be adjusted to make corrections. For example, one or more of the selected attachments may be in a wrong place and/or missing. At 1209, a final scan of the patient's dentition with the bonded attachments is optionally taken (after being adjusted in 1209). At 1211, one or more aligners are fabricated based on the scan with selected attachments bonded to the teeth.

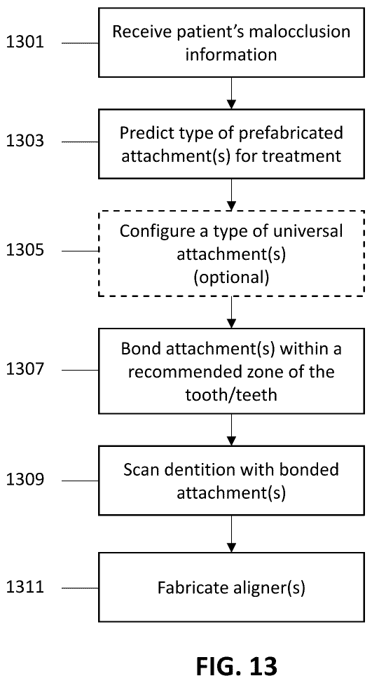

FIG. 13 is a flowchart indicating an example process using prefabricated dental attachments, where the types of attachments are predicted, and the attachments are bonded at recommended zones of the teeth. At 1301, the patient's malocclusion information (and optionally chief concerns) is received. At 1303, the type of attachments for treating the malocclusion(s) is predicted. In some cases, this may include choosing from one or more attachment types that are categorized by type of malocclusion. For example, different types of attachments may be categorized based on whether the attachment is used for treatment of class I malocclusion, class II malocclusion, class III malocclusion, crowding, overbite, underbite, overjet, crossbite, open bite, gapped teeth, or some combination of these malocclusions. The categories of the attachments may alternatively or additionally be categorized based on the magnitude and/or direction of tooth movement, type of tooth movement (e.g., translation, rotation, intrusion, or extrusion), and/or type of tooth (e.g., molar, premolar, incisor, or canine). For example, if the plan is to extrude a left upper central tooth, the selected attachment may be categorized as an extrusion attachment for this tooth, and the best location for this purpose may be calculated and designated.

Alternatively or additionally, predicting the type of attachments may include determining a target configuration of the teeth, determining forces to apply on the teeth to accomplish the target configuration (e.g., according to stages of a treatment plan), and estimating which type of attachment(s) would best be used to apply the forces. In some cases, artificial intelligence (AI) may be used to predict what type of attachment would be needed for which teeth. For example, one or more particular types of attachments on one or more upper anterior teeth may be predicted for treatment of a severe deep bite and lowering smile-line, which may require an upper central extrusion and lower anterior intrusion.

At 1305, types of one or more prefabricated (off-the-shelf) attachments may optionally be configured. These attachments may have predetermined characteristics, and not be customized or optimized for treatment of the particular patient. As discussed previously, this may allow better control of unwanted tooth movement, provide more options for greater range of tooth movement, and/or allow for more complex movements. This may also be useful in cases when the attachment requires re-bonding with an aligner (e.g., without a template) when attachment debonding occurs. In some examples, a user interface presents the off-the-shelf attachment(s) to the user, for example, for purchase. In some cases, the user interface presents an option for subscription of the off-the-shelf attachment(s).

At 1307, the attachment(s) are bonded within a recommended zone of the tooth/teeth, as discussed. Note that the actual bonding location of the attachment(s) may not require complete accuracy since a scan of the dentition is made with the bonded attachment(s).

At 1309, the dentition with the bonded attachment(s) is scanned. These images may be used to create a virtual 3D model of the dentition with the bonded attachment(s). The virtual 3D model may be segmented to distinguish between teeth, attachment(s), and/or gingiva. In some examples, the scan data and/or virtual 3D model may be sent from a dental practitioner's office to the aligner manufacturer.

At 1311, one or more aligners are fabricated based on a virtual model created from the scanned dentition with attachment(s). The aligner(s) may then be sent to the dental practitioner and given to the patient for initiating treatment.

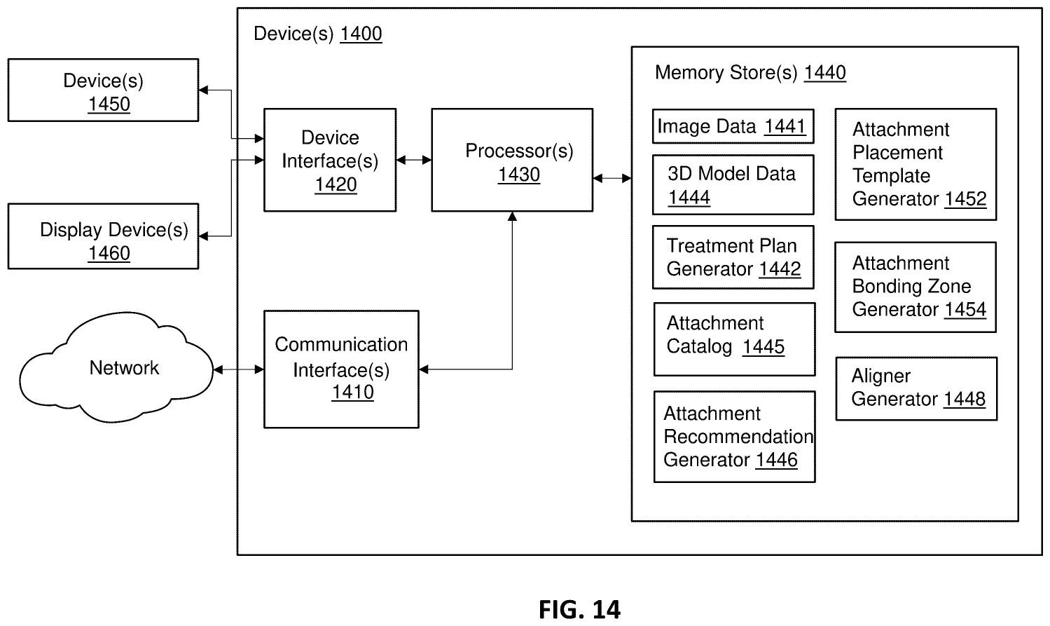

FIG. 14 shows an example system that includes one or more devices 1400 for implementing one or more of the methods described herein. Although described herein as a device, the functionality of the device 1400 may be performed by any feasible apparatus, system, or method. The device 1400 may include one or more communication interfaces 1410, one or more device interfaces 1420, one or more processors 1430, and one or more memory stores 1440. In some examples, the device 1400 may be configured to analyze images (e.g., dental images) and generate output resulting from the analysis.

The communication interface 1410, which may be coupled to a network and to the processor 1430, may transmit signals to and receive signals from other wired or wireless devices, including remote (e.g., cloud-based) storage devices, cameras, and/or displays. For example, the communication interface 1410 may include wired (e.g., serial, ethernet, or the like) and/or wireless (Bluetooth, Wi-Fi, cellular, or the like) transceivers that may communicate with any other feasible device through any feasible network.

The device interface 1420, which is coupled to the processor 1430, may be used to interface with any feasible input and/or output device. The device(s) 1450 may include one or more image capturing devices. Example image capturing devices may include intraoral scanners, optical cameras, x-ray devices, panoramic x-ray devices, portable cameras (e.g., phone cameras), and/or other imaging devices. In some examples, the device interface 1420 may be coupled to an interface with a display device 1460. The device(s) 1450 may include one or more fabrication devices. Example fabrication devices may include one or more 3D printers, molding devices, and/or other manufacturing devices. Through the display device 1460 (e.g., computer monitor), the processor 1430 may display images, feedback information, instructions, or the like.

In some examples, the image capturing device 1450 and/or the display device 1460 may be an integral part of the device 1400. In other words, the image capturing device 1450 and/or the display device 1460 may share a common housing or enclosure. For example, the device 1400 may be a cell phone, a tablet computer, or a laptop computer that includes at least these elements. In some examples, the image capturing device 1450 and/or the display device 1460 are physically separate from the device 1400.

The processor 1430, which is coupled to the memory store 1440, may be any one or more suitable processors capable of executing scripts or instructions of one or more software programs stored in the device 1400 (such as within the memory store 1440).

The memory store 1440 may include a non-transitory computer-readable storage medium (e.g., one or more nonvolatile memory elements, such as EPROM, EEPROM, Flash memory, a hard drive, etc.).

The memory store 1440 may include image data 1441. The image data 1441 may include one or more images (e.g., 2D images), for example, captured by the one or more imaging capturing devices 1450. For example, the image data 1441 may be obtained through the communication interface 1410 and stored within the memory store 1440. The image data 1441 may include dental images, optical images, x-ray images, panoramic images, video images, video frames, composite images formed from two or more source images, and the like. The image data 1441 may include segmented data, in which boundaries of objects (e.g., teeth and/or gums) are identified and labeled (e.g., on a pixel-by-pixel basis). In some examples, the processor 1430 is configured to segment an unsegmented image.

The memory store 1440 may include 3D model data 1444. The 3D model data 1444 may include one or more digital 3D models each represented as a mesh. For example, the 3D model data 1444 may include a series of digital 3D models represented as a series of meshes corresponding to different stages of a treatment plan for a patient. The treatment plan may be generated by a treatment plan generator 1442 that is executable by the processor 1430. The 3D models may be segmented such that boundaries of objects (e.g., teeth and/or gums) are identified and labeled. In some examples, the 3D models are imported (e.g., via the communication interface 1410) as pre-segmented meshes. In other examples, the processor 1430 is configured to generate the 3D models. For example, the processor 1430 may be configured to generate meshes from 3D image data, for example, from the image data 1441 (e.g., from a scan of a patient's teeth). In some examples, the 3D model is generated based on images of the patient's teeth with attachments bonded to the teeth.

The memory store 1440 may include an attachment catalogue 1445 that includes multiple attachments having different characteristics (e.g., shape, size, rigidity, elasticity, deformability, material, and/or color). The attachments may be categorized by one or more of the characteristics and/or by usage case. Usage cases may include the type of malocclusion that the attachment may be useful in treating, the magnitude and/or direction of tooth movement, and/or type of tooth movement.

An attachment recommendation generator 1446 may be executable by the processor 1430 to generate an attachment recommendation. The attachment recommendation may be based on the type of malocclusion(s), chief concerns, target tooth configuration, and/or other factors. In some cases, the attachment recommendation may be based on the treatment plan (e.g., initial treatment plan).

An attachment placement template generator 1452 may be executable by the processor 1430 to generate one or more virtual attachment placement templates (e.g., digital 3D models). The attachment placement template generator 1452 may determine a shape of the virtual attachment placement template to couple with selected one or more off-the-shelf attachments and position the selected one or more off-the-shelf attachments with respect to bonding locations on the teeth based on the treatment plan. A virtual attachment placement template(s) may be in a format that is capable of being read by a fabrication device (e.g., device 1450) to produce one or more attachment placement template device.

An attachment bonding zone generator 1454 may be executable by the processor 1430 to generate a recommended bonding zone on one or more teeth of the patient. The recommended bonding zone may be determined based on the digital 3D model of the patient's teeth. In some examples, the recommended bonding zone may be based on clinical aspects of the teeth, such as FACC and/or FA points. In some examples, the recommended bonding zone may be used instead of an attachment placement template device.

An aligner generator 1448 may be executable by the processor 1430 to generate one or more virtual aligners (e.g., one or more digital 3D models). A shape of a virtual aligner may be based on the treatment plan. The virtual aligner may be shaped to apply forces on the one or more off-the-shelf attachments bonded on the patient's teeth in directions and magnitudes according to the treatment plan. For example, a set of virtual aligners where each virtual aligner is configured to implement a stage of the treatment plan may be generated. The virtual aligner(s) may be in a format that is capable of being read by a fabrication device (e.g., device 1450) to produce one or more aligners.

The systems described herein may be used to scan (e.g., using an intraoral scanner of the system) and/or analyze a scan of a patient's teeth. As described herein, a scanner may be configured to obtain images of the patient's teeth with or without attachments bonded to them. In some cases, a scan may be performed to determine whether all attachments have been correctly placed on the patient teeth in accordance with one or more aligners that have already been manufactured (or will be manufactured). If the system determines that the attachments have not been correctly places, the system may be configured to provide a recommendation to move one or more attachments, use one or more different attachments, remove one or more attachments, and/or leave the attachments as is and inform the user that the final outcome may be compromised.

### Systems

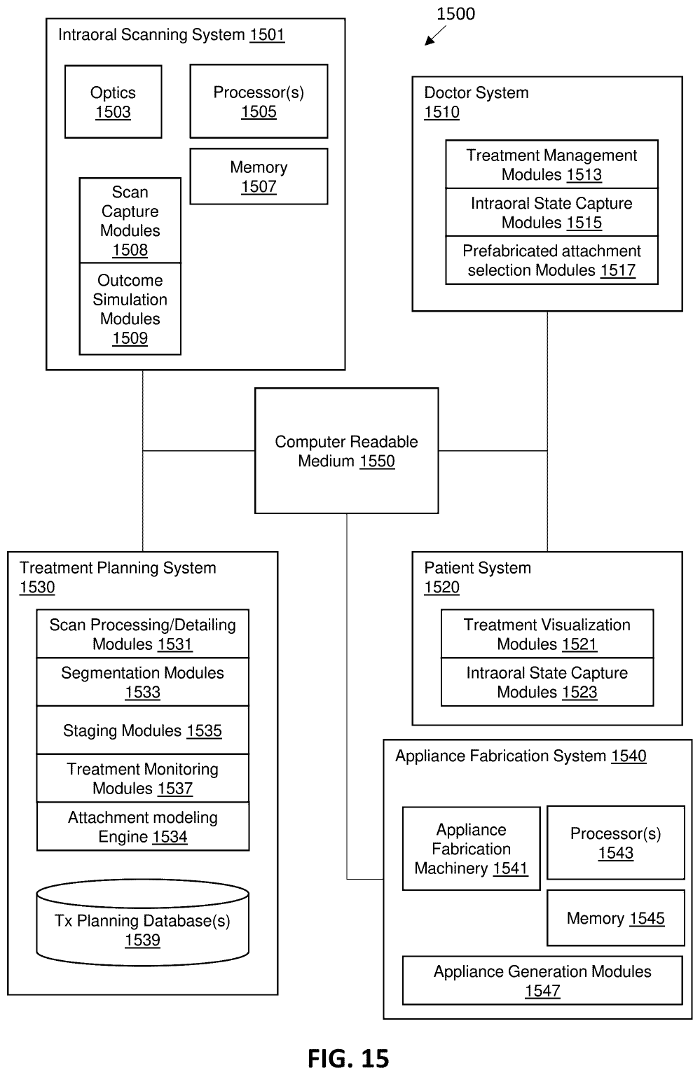

In general, these methods and apparatuses may be used at one or more parts of a dental computing environment, including as part of an intraoral scanning system, doctor system, treatment planning system, patient system, and/or fabrication system. In particular, these methods and apparatuses may be used as part of a treatment planning system, for example, to generate and/or modify one or more treatment plans, and/or for generating one or more dental appliances to perform the treatment plan. For example, FIG. 15 is a diagram illustrating one variation of a computing environment 1500 that may generate one or more orthodontic treatment plans specific to a patient, and fabricate dental appliances that may accomplish the treatment plan to treat a patient, under the direction of a dental professional, using the prefabricated attachments described herein. The example computing environment 1500 shown in FIG. 15 may include an intraoral scanning system 1501, a doctor system 1510, a treatment planning system 1530 (e.g., technician system), a patient system 1520, an appliance fabrication system 1540, and computer-readable medium 1550. In some variations a computing environment (dental computing system) 1500 may include just one or a subset of these systems (which may also be referred to as sub-systems of the overall system 1500). Further, one or more of these systems may be combined or integrated with one or more of the other systems (sub-systems), such as, e.g., the patient system and the doctor system may be part of a remote server accessible by doctor and/or patient interfaces. The computer readable medium 960 may divided between all or some of the systems (subsystems); for example, the treatment planning system and appliance fabrication system may be part of the same sub-system and may be on a computer readable medium 1550. Further, each of these systems may be further divided into sub-systems or components that may be physically distributed (e.g., between local and remote processors, etc.) or may be integrated.

An intraoral scanning system may include an intraoral scanner as well as one or more processors for processing images. For example, an intraoral scanning system 1501 can include optics 1503 (e.g., lens(es), filters, light sources, etc.), processor(s) 1505, a memory 1507, scan capture modules 1508, and outcome simulation modules 1509. In general, the intraoral scanning system 1501 can capture one or more images of a patient's dentition. Use of the intraoral scanning system 1501 may be in a clinical setting (doctor's office or the like) or in a patient-selected setting (the patient's home, for example). In some cases, operations of the intraoral scanning system 1501 may be performed by an intraoral scanner, dental camera, cell phone or any other feasible device.

Any of the component systems or sub-systems of the dental computing environment 1500 may access or use the prefabricated attachment methods and apparatuses described herein. For example, the doctor system 1510 may include treatment management modules 1513 and intraoral state capture modules 1515 that may access or use both the 3D model of the patient's dentition and one or more digital models of the prefabricated attachments. The doctor system 1510 may provide a “doctor facing” interface to the computing environment 1500. In some examples the doctor system may include one or more prefabricated attachment selection modules 1517 to allow the dental professional (e.g., doctor) to select or confirm selection of one or more prefabricated attachments; the prefabricated attachment selection module may allow the dental profession to choose one or more prefabricated attachments as described herein and/or it may allow the dental professional to confirm the user of one or more prefabricated attachments as suggested or modeled by the (prefabricated) attachment modeling engine 1534, which may be part of the Treatment planning system 1530, as shown.

The treatment management modules 1513 can perform any operations that enable a doctor or other clinician to manage the treatment of any patient. In some examples, the treatment management modules 1513 may provide a visualization and/or simulation of the patient's dentition with respect to a treatment plan.

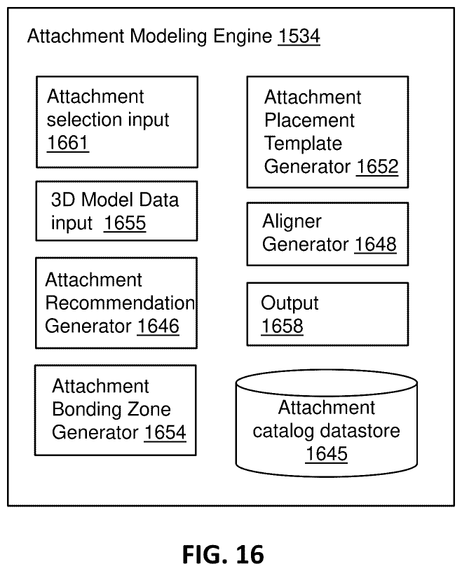

In some examples, the doctor system may include a user interface for the doctor that allows the doctor to interactively select one or more prefabricated attachment using the prefabricated attachment module 1517, which may access a database/data store (or catalog) of prefabricated attachments, and may allow the user to select among these. The prefabricated attachment module may then send the selected prefabricated attachments to the treatment planning system 1530 to determine how to implement the treatment plan using the selected attachments (e.g., using the attachment modeling engine 1535, and see FIG. 16). Alternatively, the attachment modeling engine may recommend one or more prefabricated attachments to the dental professional, e.g., through the doctor system 1510.

The intraoral state capture modules 1515 can provide images of the patient's dentition to a clinician through the doctor system 1510. The images may be captured through the intraoral scanning system 1501 and may also include images of a simulation of tooth movement based on a treatment plan, including movement using the prefabricated attachments.

In some examples, the treatment management modules 1513 can enable the doctor to modify or revise a treatment plan, particularly when images provided by the intraoral state capture modules 1515 indicate that the movement of the patient's teeth may not be according to the treatment plan. The doctor system 1510 may include one or more processors configured to execute any feasible non-transitory computer-readable instructions to perform any feasible operations described herein.

Alternatively or additionally, the treatment planning system 1530 may include any of the methods and apparatuses described herein, including implemented the user of the one or more prefabricated attachments using the prefabricated attachment engine 1534. The treatment planning system 1530 may include scan processing/detailing modules 1531, segmentation modules 1533, staging modules 1535, treatment monitoring modules 1537, and treatment planning database(s) 1539. In general, the treatment planning system 1530 can determine a treatment plan for any feasible patient. The scan processing/detailing modules 1531 can receive or obtain dental scans (such as scans from the intraoral scanning system 1501) and can process the scans to remove scan errors and, in some cases, enhancing details of the scanned image.

A treatment planning system may include a segmentation modules 1533 that can segment a dental model into separate parts including separate teeth, gums, jaw bones, and the like. The staging modules 1535 may determine different stages of a treatment plan. Each stage may correspond to a different dental aligner and may use the same or different attachments. The staging modules 1535 may also determine the final position of the patient's teeth, in accordance with a treatment plan. Thus, the staging modules 1535 can determine some or all of a patient's orthodontic treatment plan. In some examples, the staging modules can simulate movement of a patient's teeth in accordance with the different stages of the patient's treatment plan using the determined attachments.

The treatment monitoring modules 1537 can monitor the progress of an orthodontic treatment plan. In some examples, the treatment monitoring modules can provide an analysis of progress of treatment plans to a clinician. The orthodontic treatment plans may be stored in the treatment planning database(s) 1539. Although not shown, the treatment planning system 1530 can include one or more processors configured to execute any feasible non-transitory computer-readable instructions to perform any feasible operations described herein.

The patient system 1520 can include treatment visualization modules 1521 and intraoral state capture modules 1523. In general, the patient system can provide a “patient facing” interface to the computing environment 1500. The treatment visualization modules 1521 can enable the patient to visualize how an orthodontic treatment plan has progressed and also visualize a predicted outcome (e.g., a final position of teeth). In some examples, the patient system 1520 can capture dentition scans for the treatment visualization modules 1521 through the intraoral state capture modules 1523. The intraoral state capture modules can enable a patient to capture his or her own dentition through the intraoral scanning system 1501. Although not shown here, the patient system 1520 can include one or more processors configured to execute any feasible non-transitory computer-readable instructions to perform any feasible operations described herein.

The appliance fabrication system 1540 can include appliance fabrication machinery 1541, processor(s) 1543, memory 1545, and appliance generation modules 1547. In general, the appliance fabrication system 1520 can directly or indirectly fabricate aligners to implement an orthodontic treatment plan. In some examples, the orthodontic treatment plan may be stored in the treatment planning database(s) 1539.

The appliance fabrication machinery 1541 may include any feasible implement or apparatus that can fabricate any suitable dental aligner. The appliance generation modules 1547 may include any non-transitory computer-readable instructions that, when executed by the processor(s), can direct the appliance fabrication machinery 1541 to produce one or more dental aligners. The memory 1545 may store data or instructions for use by the processor(s) 1543. In some examples, the memory 1545 may temporarily store a treatment plan, dental models, or intraoral scans.

The computer-readable medium 1550 may include some or all of the elements described herein with respect to the computing environment 1500. The computer-readable medium 1550 may include non-transitory computer-readable instructions that, when executed by a processor, can provide the functionality of any device, machine, or module described herein.

FIG. 16 schematically illustrates one example of an attachment modeling engine 1534, as mentioned above. In this example the attachment modeling engine may include one or more input modules, such as an attachment selection input 1661, for receiving one or more selections for prefabricated attachments, and/or confirming suggested prefabricated attachments (e.g., from an attachment recommendation generator 1646). The attachment modeling engine may also include one or more inputs, e.g., a 3D model data input, for receiving the 3D model of the patient's dentition.

The attachment modeling engine 1534 may also include an attachment bonding zone generator 1654 and an attachment catalog datastore 1645 (e.g., including information about the available prefabricated attachments, such as digital models of these prefabricated attachments).

As discussed above, he attachment modeling engine may also include an attachment placement template generator 1652 for generating a template (e.g., jig) for applying the prefabricated attachments to a particular patient's dentition.

The attachment modeling engine may include or may be configured to include or may communicate with an aligner generator 1648 configured to generate or modify the dental appliance(s) to implement the treatment plan and include mating sites for the prefabricated attachments. The attachment modeling engine may also include one or more outputs 1658 for outputting the prefabricated attachments, modified dental appliance(s), jig, etc.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of forming a series of aligners for orthodontic treatment of a patient's dentition, the method comprising:
accessing a library of prefabricated dental attachments, wherein the prefabricated dental attachments have predetermined characteristics;
generating an orthodontic treatment plan for moving the patient's teeth from an initial configuration toward a target configuration by applying repositioning forces to the patient's teeth according to a series of treatment stages;
selecting one or more prefabricated dental attachments from the library of prefabricated dental attachments for applying the repositioning forces to the patient's teeth according to the series of treatment stages;
determining a shape of each aligner of the series of aligners, wherein each aligner is configured to apply repositioning forces to the patient's teeth according to a corresponding stage of the orthodontic treatment plan, further wherein one or more of the aligners includes one or more attachment cavities to accommodate the selected one or more prefabricated dental attachments; and
causing the series of aligners to be fabricated based on the determined shapes.

2. The method of claim 1, wherein the prefabricated dental attachments have different characteristics including one or more of: a shape, a size, a rigidity, an elasticity, a deformability, and a material.

3. The method of claim 1, further comprising providing a recommendation as to which of the prefabricated dental attachments to select from the library based at least partially on the initial configuration and the target configuration of the patient's teeth.

4. The method of claim 1, further comprising displaying a user interface that allows a user to enter one or more types of malocclusions, one or more chief concerns, or one or more types of malocclusions and one or more chief concerns, and further comprising providing a recommendation as to which of the prefabricated dental attachments to select based at least partially on information entered in the user interface.

5. The method of claim 1, wherein the one or more prefabricated attachments are configured to be bonded at one more predetermined locations on the patient's teeth.

6. The method of claim 1, further comprising receiving a scan of the patient's teeth with the selected one or more prefabricated dental attachments bonded to the patient's teeth, wherein the shape of each aligner is based on the scan.

7. The method of claim 1, further comprising receiving a scan of the patient's teeth without the selected one or more prefabricated dental attachments bonded to the patient's teeth, wherein the orthodontic treatment plan is generated based on the scan.

8. The method of claim 1, further comprising determining attachment bonding zones corresponding to approximate bonding locations of the selected one or more dental attachments on the patient's teeth, and providing the attachment bonding zones to a user.

9. The method of claim 1, wherein causing the series of aligner to be fabricated comprises transmitting instructions for fabricating the series of aligners based on the determined shape of each aligner.

10. The method of claim 1, wherein causing the series of aligners to be fabricated comprises fabricating the series of aligners based on the instructions using by direct fabrication or a molding process.

11. The method of claim 1, further comprising generating a virtual attachment placement template device having a shape configured to fit on the patient's teeth and position the selected one or more prefabricated dental attachments at corresponding bonding locations of the patient's teeth.

12. The method of claim 11, further comprising transmitting instructions for fabricating an attachment placement template device based on the virtual attachment placement template device, wherein the attachment placement template device includes the selected one or more prefabricated dental attachment attached thereto.

13. A system for forming a series of aligners for orthodontic treatment of a patient's dentition, the system comprising:
one or more processors; and
one or more memory stores coupled to one or more processors, the one or more memory stores configured to store computer instructions that, when executed by the one or more processors, perform a computer-implemented method comprising:
accessing a library of prefabricated dental attachments, wherein the prefabricated dental attachments have predetermined characteristics;
generating an orthodontic treatment plan for moving the patient's teeth from an initial configuration toward a target configuration by applying repositioning forces to the patient's teeth according to a series of treatment stages;
selecting one or more prefabricated dental attachments from the library of prefabricated dental attachments for applying the repositioning forces to the patient's teeth according to the series of treatment stages;
determining a shape of each aligner of the series of aligners, wherein each aligner is configured to apply repositioning forces to the patient's teeth according to a corresponding stage of the orthodontic treatment plan, further wherein one or more of the aligners includes one or more attachment cavities to accommodate the selected one or more prefabricated dental attachments; and
causing the series of aligners to be fabricated based on the determined shapes.

14. The system of claim 13, wherein the computer-implemented method further comprises providing a recommendation as to which of the prefabricated dental attachments from the library to select based at least partially on the initial configuration and the target configuration of the patient's teeth.

15. The system of claim 13, wherein the computer-implemented method further comprises:
displaying a user interface that allows a user to enter one or more types of malocclusions, one or more chief concerns, or one or more types of malocclusions and one or more chief concerns; and
providing a recommendation as to which of the prefabricated dental attachments from the library to select based at least partially on information entered in the user interface.

16. The system of claim 13, wherein the one or more prefabricated attachments are configured to be bonded at one more predetermined locations on the patient's teeth.

17. The system of claim 13, wherein the prefabricated dental attachments have different characteristics including one or more of: a shape, a size, a rigidity, an elasticity, a deformability, a material, and a color.

18. The system of claim 13, wherein the computer-implemented method further comprises receiving a scan of the patient's teeth with the selected one or more prefabricated dental attachments bonded to the patient's teeth, wherein the shape of each aligner is based on the scan.

19. The system of claim 13, wherein the computer-implemented method further comprises receiving a scan of the patient's teeth without the selected prefabricated one or more dental attachments bonded to the patient's teeth, wherein the orthodontic treatment plan is generated based on the scan.

20. The system of claim 13, wherein the computer-implemented method further comprises generating a virtual attachment placement template device having a shape configured to fit on the patient's teeth and position the selected one or more prefabricated dental attachments at corresponding bonding locations of the patient's teeth.

21. The system of claim 20, wherein the computer-implemented method further comprises transmitting instructions for fabricating an attachment placement template device based on the virtual attachment placement template device, wherein the attachment placement template device includes the selected prefabricated one or more dental attachment attached thereto.

22.-43. (canceled)
